(19) United States
(12) Patent Application Publication
(10) Pub. No.: US 2016/0271344 A1
(43) Pub. Date: Sep. 22, 2016

(54) SYSTEM FOR THE VAPORIZATION AND DELIVERY OF LIQUIDS

(71) Applicant: Victor Wong, Austin, TX (US)

(72) Inventor: Victor Wong, Austin, TX (US)

(21) Appl. No.: 14/663,090

(22) Filed: Mar. 19, 2015

(51) Int. Cl. A61M 11/02

(52) U.S. Cl. CPC A61M 11/02

(57) ABSTRACT

A system for the vaporization and delivery of liquids. The invention allows for the vaporization of liquids in an easy and economical way that does not require the manufacturing apparatuses to be present at the time of delivery.

### PRIORITY

The present invention claims priority under 35 USC section 119 based upon a provisional application with a Ser. No. of 61/955,208 which was filed on Mar. 19, 2014.

### FIELD OF THE INVENTION

The present invention relates to the method and apparatus for the vaporization of liquids.

### BACKGROUND OF THE INVENTION

Existing methods for vaporization of liquids require significant apparatuses for both manufacturing and delivery and/or the manufacturing system must be present at the point of delivery. Past vaporization employs heat which may be hazardous and may cause harm, or the products require a continual stream of vaporized alcohol, increasing the chance for alcohol poisoning. Other products may require a greater amount of alcohol causing the blood level of alcohol to exceed legal limits.

### SUMMARY

The present invention minimizes the complexity and costs of achieving vaporization. In addition, the present invention allows for the delivery at a time and place separate from the manufacturing.

A method for vaporization of a liquid may include the steps of placing the liquid in a container, adding a gas to the liquid in the container, pressurizing the gas and liquid in the container and opening the container rapidly to vaporize the gas and liquid together.

The method may additionally include the step of sealing the container after the gas and liquid have been pressurized.

The method may additionally include the step of breaking the seal in the container to vaporize the gas and liquid together.

The method may additionally include the step of pressurizing the gas and liquid with a pressure vessel.

The method may additionally include the step of pressurizing the gas and liquid with a solid-state gas.

The solid-state gas may be dry ice.

The liquid may be alcohol.

The liquid may be medication.

The method may additionally include the step of inhaling the vaporization by a user.

### BRIEF DESCRIPTION OF THE DRAWINGS

The invention may be understood by reference to the following description taken in conjunction with the accompanying drawings, in which, like reference numerals identify like elements, and in which:

FIG. 1a illustrates the container of the present invention;

FIG. 1b illustrates the cap of the present invention;

FIG. 2a illustrates another container of the present invention;

FIG. 2b illustrates another cap of the present invention;

FIG. 3 illustrates FIG. 3a and FIG. 3b;

FIG. 3a illustrates a production method of the present invention;

FIG. 3b illustrates a use method of the present invention;

FIG. 4 illustrates a another embodiment of the present invention;

FIG. 5 illustrates another embodiment of the present invention.

### DETAILED DESCRIPTION OF THE INVENTION

As stated above, existing systems for vaporization of liquids require significant apparatuses for both manufacturing and delivery.

The present invention described here solves this problem.

The invention provides rapid vaporization of liquids by a device for pressurization and rapid depressurization in an inexpensive sealed container.

### THE INVENTION DISCUSSED HERE INCLUDES

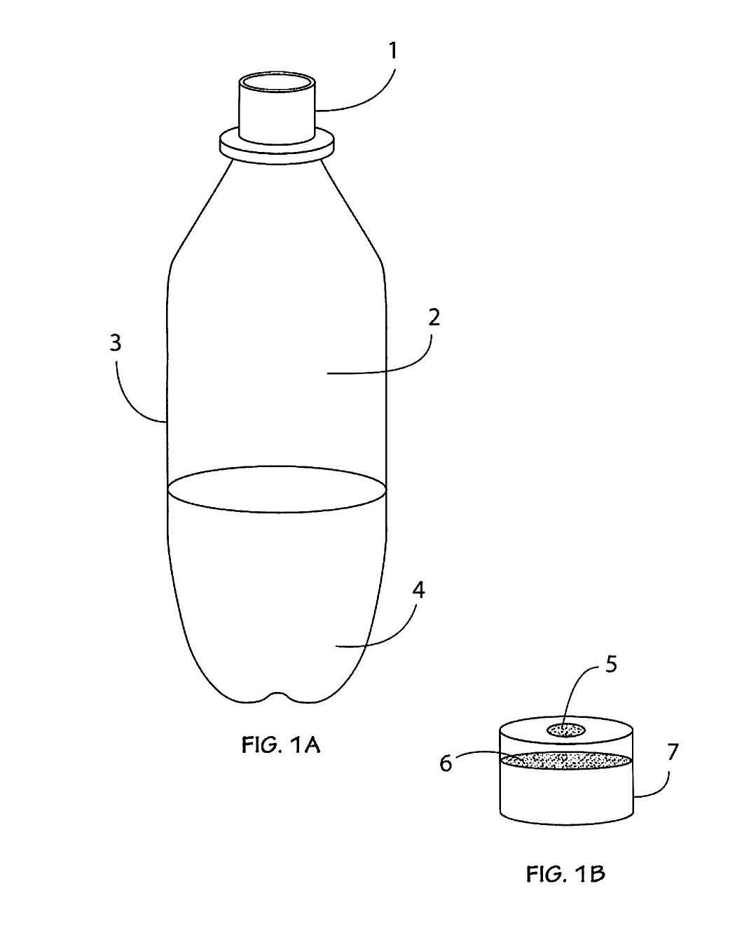

(FIG. 1a,b)

1. Cap assembly

2. Gas

3. Container

4. Liquid

5. Fill Valve

6. Seal

7. Cap

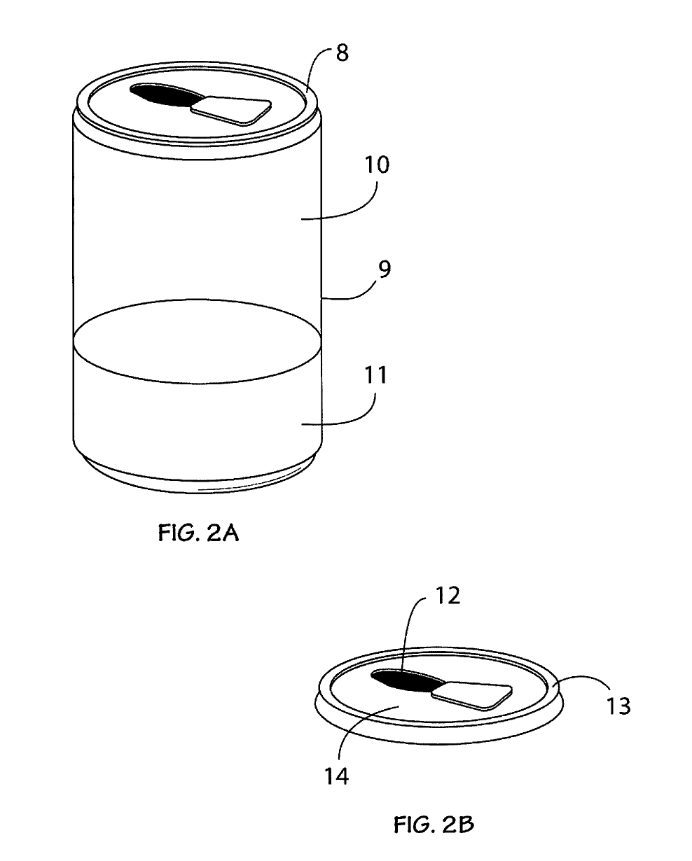

(FIG. 2a,b)

8. Cap Assembly

9. Container

10. Gas

11. Liquid

12. Release Valve

13. Seal

14. Cap

The present invention provides a low-cost and easy method to vaporize liquids for personal enjoyment or medical use in delivering medication to a user. Since the process is highly efficient, a fraction of the material is needed versus introduction of the material into the body through oral ingestion. For alcohol, an amount less than 1/60 of a traditional liquid shot is needed to achieve the desired effect, thereby reducing the negative effects of drinking alcohol while still providing a pleasant feeling. The duration of each dose is also much lower at approximately 15 minutes, thereby allowing a person to enjoy the effects of alcohol with a greatly reduced chance of a hangover. Other possible social benefits of this method may prevent drunk driving thereby saving life. The reduction of the actual material will greatly decrease the cost for medicine also.

### Relationships Between The Components:

In FIG. 1a,b—The Cap assembly (1) includes the Fill valve (5) to allow the container 3 to be pressurized with gas, the Seal (6) to seal the liquid and gas within the container 3 and the Cap (7) to connect to the container 3. The fill valve 5 may be a one-way valve to prevent gas or liquid from escaping the container 3 through the valve 5. The Cap assembly (1) may be attached to the container (3) with threads or other attachment devices, which is filled with a Liquid (4) and a Gas (2).

In FIG. 2a,b (An alternate structure of the invention)—The Cap assembly (8) may include the Release value (12), the Seal (13) and the Cap (14). The Cap assembly (8) may be attached to the container (9), which is filled with a Liquid (11) and a Gas (10).

### How the Invention Works:

The liquid vaporization process in the invention is the rapid change almost instantaneous in pressure within a container for example when the entire cap is instantaneously removed without hesitation. Existing containers only allow graduate pressure changes, which will not cause the liquid to vaporize.

In the invention, the Seal (6) or the Release valve (12) provides a rapid release of pressure needed for the vaporization process by breaking the seal 6 or the release valve 12 simultaneously around the periphery of the seal 6 or the release valve 12.

In the preferred embodiment using FIG. 1a,b, the container 3 is filled with Liquid (4) and then the Cap (1) is attached. The Container (3) is then pressurized by an external device with Gas (2) or with a pre-mixed combination of Gas (2) and Liquid (4), injected into the Container (3) via the Fill valve (5) of the Cap (7), which has already been attached. This constitutes the completion of the manufacturing process. To vaporize the liquid, the Cap (1) is opened releasing Seal (6) causing a rapid pressure change. The vaporized liquids is now ready for delivery.

One application of the invention is to vaporize ethyl alcohol, optionally including flavor or aroma ingredients, for personal consumption.

### How to Make the Invention

For FIG. 1a,b; Embed the Fill valve (5) and Seal (6) into the Cap (7). Fill the container (3) with Liquid (4). Attach the Cap (1) and then pressurize with Gas (2) through Fill valve (5). As an alternative to prefilling the Container (3) with Liquid (4) before sealing the container with Cap (1), the Liquid (4) may be filled through the Fill valve (5) independently or in combination with the Gas (2).

For FIG. 2a,b: Produce the Cap (14) containing the Release valve (12). Fill the Container (9) with Liquid (11). The Cap (8) must then be attached to Container (9) using Seal (13) while Gas (10) is already under pressure. The container 9 may be formed from metal, plastic, glass or other appropriate material.

### How to use the Invention

The user opens the Cap (1) or Release value (12) of the pressurized Container (3) or (9) as described above, thereby rapidly releasing the pressure and vaporizing the liquid contents. The vaporized liquid is now ready for delivery to the user.

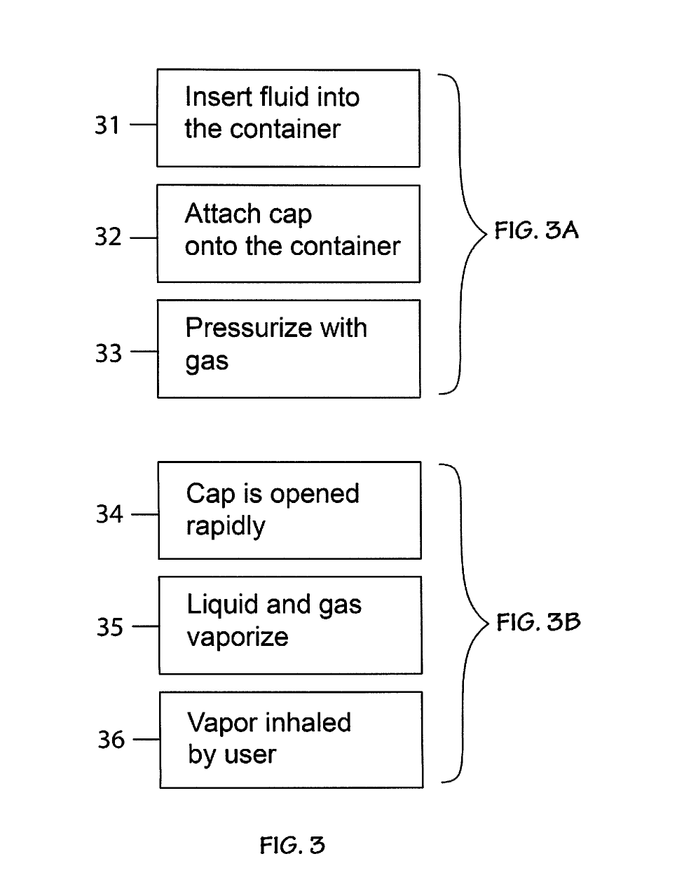

FIG. 3a illustrates the steps of production for the present invention which may include step 31 to insert fluid into the container, step 32 to attach the cap on to the container and step 33 to pressurized the container with gas.

FIG. 3b illustrates the steps of usage for the present invention which may include an step 34 to open the cap rapidly, step 35 to vaporize the liquid and the gas and step 362 to inhale the vapor by the user.

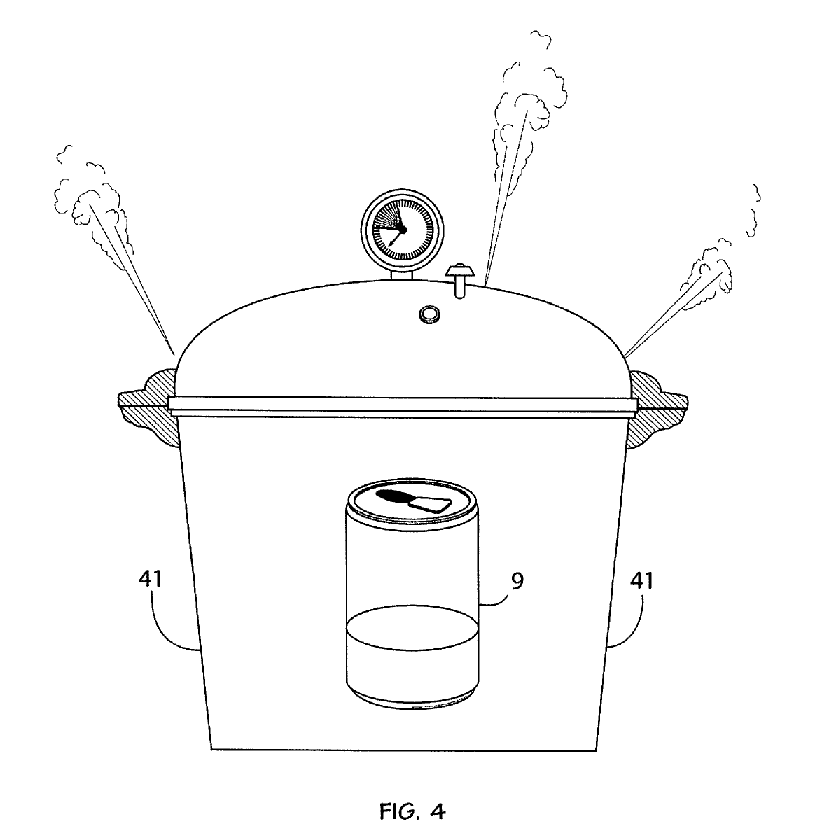

FIG. 4 illustrates an alternative device/method to pressurize the container which may be placed inside a pressure vessel 41 which may be sealed and pressurized to place the container 3, 9 under pressure. More particularly, the entire container 3, 9 is placed into the pressure vessel 41 or another type of mechanism which may only but completely cover the opening of the container 3, 9 to inject pressure and liquid into the container 3, 9. Subsequently, the container 3, 9 is sealed. This may be applicable to single use containers 3, 9 where the product may be sold by a 3^{rd} party such as a liquor store. Additionally, this application may be applicable to single dose medication which may be delivered by inhaling the vaporization into the lungs of the user.

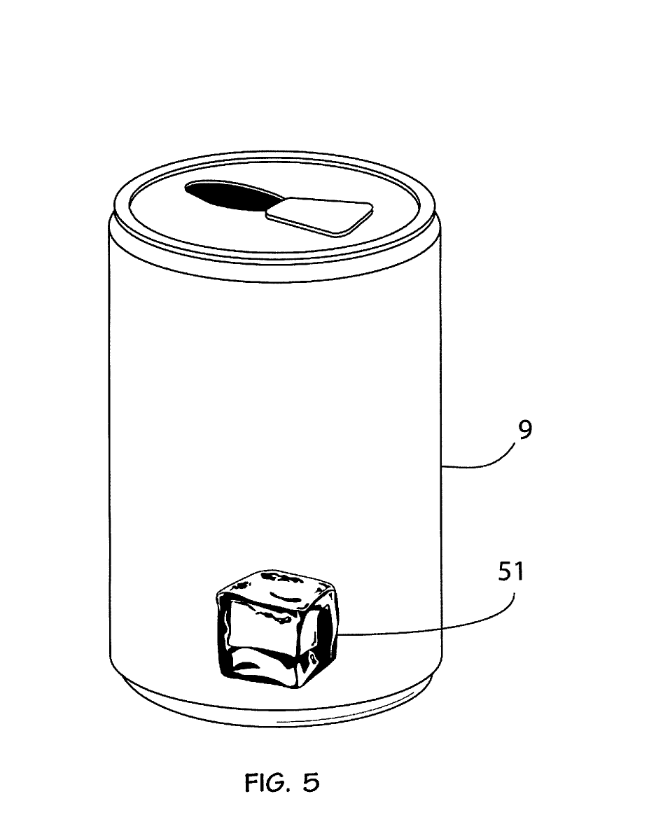

FIG. 5 illustrates another alternative to pressurizing the container 3, 9 by placing the liquid into the container 3, 9 along with a solid-state gas such as dry ice 51. The container 3, 9 may be sealed and as the dry ice 51 melts, pressure will be created in the container 3, 9 as the dry ice 51 turns to gas.

1) A method for vaporization of a liquid, comprising the steps of:
placing the liquid in a container;
adding a gas to the liquid in the container;
pressurizing the gas and liquid in the container;
opening the container rapidly to vaporize the gas and liquid together;

2) A method for vaporization of a liquid as in claim 1, wherein the method additionally includes the step of sealing the container after the gas and liquid have been pressurized.

3) A method for vaporization of a liquid as in claim 2, wherein the method additionally includes the step of breaking the seal in the container to vaporize the gas and liquid together.

4) A method for vaporization of a liquid as in claim 1, wherein the method includes the step of pressurizing the gas and liquid with a pressure vessel.

5) A method for vaporization of a liquid as in claim 1, wherein the method includes the step of pressurizing the gas and liquid with a solid-state gas.

6) A method for vaporization of a liquid as in claim 5, wherein the solid-state gas is dry ice.

7) A method for vaporization of a liquid as in claim 1, wherein the liquid is alcohol.

8) A method for vaporization of a liquid as in claim 1, wherein the liquid is medication.

9) A method for vaporization of a liquid as in claim 1, wherein the method further includes the step of inhaling the vaporization by a user.
